(19) United States
(12) Patent Application Publication
(10) Pub. No.: US 2002/0016512 A1
(43) Pub. Date: Feb. 7, 2002

(54) Heterogeneously catalyzed process for cross coupling aryl chlorides with aryl boronic acids

(76) Inventors: Yongkui Sun, Bridgewater, NJ (US); Carl LeBlond, Somerset, NJ (US); John R. Sowa JR., Morristown, NJ (US)

Correspondence Address: MERCK AND CO INC, P O BOX 2000, RAHWAY, NJ, 070650907

(21) Appl. No.: 09/906,244

(22) Filed: Jul. 16, 2001

Related U.S. Application Data
(60) Provisional application No. 60/218,990, filed on Jul. 17, 2000.

(51) Int. Cl. C07D 213/46; C07C 45/46

(52) U.S. Cl. 568/323; 546/314

(57) ABSTRACT

This invention relates to a ligandless process wherein heterogeneous Pd catalysts are used to activate aryl and heteroaryl chlorides for cross coupling aryl and heteroaryl chlorides and boronic acids. The process provides for use with either electron-withdrawing or electron-donating substituents, for cross coupling with boronic acids.

### BACKGROUND OF THE INVENTION

Until recently, Suzuki coupling using aryl chlorides had been a goal many are seeking to reach due to the lower cost of aryl chlorides vs. bromides and iodides. However, commonly used homogeneous Pd catalysts used for Suzuki coupling for aryl bromides such as Pd(dba)₂ or Pd(PPh₃)₄ do not work well for aryl chlorides, particularly for electron rich aryl chlorides. There has been significant progress in homogenous catalysis toward this goal. The use of strongly electron-donating phosphine ligands made it possible for the homogeneous Pd catalysts to activate the aryl chloride bond for cross coupling with boronic acids. See Xiaohong Bei, et al, Tetrahedron Lett. 1999, 40, 3855-3858; Chunming Zhang, et al, J. Org. Chem. 1999, 64, 3804-3805; John P. Wolfe, et al, J. Am. Chem. Soc. 1999, 121, 9550-9561;Adam F. Littke, et al, Angew. Chem. Int. Ed. 1998, 37, 3387-3388; David W. Old, et al, J. Am. Chem. Soc. 1998, 120, 9722 ; Fariborz Firooznia, et al. Tetrahedron Lett. 1998, 39, 3985 and Xiaohong Bei, et al, J. Org. Chem., 1999, 64, 6797. However, homogeneous catalysts are more difficult to remove and may require extra chromatographic, precipitation or extraction steps. It also requires the use of phosphine ligands, which complicates product isolation (See Japanese Patent No. 2000-336045 (Appln No. 1999-148564).

This invention relates to a process wherein heterogeneous, finely dispersed Pd catalysts are used to activate aryl chlorides for cross coupling aryl chlorides and boronic acids. The process provides for use with either electron-withdrawing or electron-donating substituents, for cross coupling with boronic acids. Unlike homogeneous catalysis, the heterogeneously catalyzed Suzuki cross-coupling process provides advantages such as ease of separation and potential re-use; ligands such as phosphine ligands are not required (ligandless process), which simplifies product isolation and eliminates side reactions that may occur between aryl groups of aryl phosphines (ligand) and the aryl boronic acid; and can result in a process with much lower cost. See also Marck G. Villeger, et al., Tetrahedron Lett. 1994, 35, 3277-3280; Gala, D., et al Org. Proc. Res. Dev. 1997, 1, 163-164; V. V. Bykov, et al, Russian Chemical Bulletin 1997, 46, 1344 and David S. Ennis, et al, Org. Proc. Res. Dev. 1999, 3, 248

### SUMMARY OF THE INVENTION

In one aspect of this invention, a ligandless process for cross-coupling aryl or heteroaryl chlorides with aryl or heteroaryl boronic acids, comprising combining an aryl or heteroaryl chloride and an aryl or heteroaryl boronic acid with a polar aprotic solvent in the presence of a base and heterogeneous palladium catalyst to produce the resulting coupled compound is disclosed.

In still another aspect of the invention, a process for synthesizing a compound of formula I:

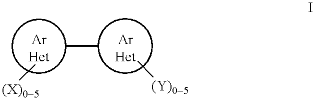

is disclosed wherein:

Ar represents C₆₋₁₀ aryl;

Het represents C₅₋₁₀ heteroaryl;

X and Y independently represent hydrogen, CF₃, C₁₋₆ alkoxy, NO₂, CN, halo, C₁₋₆ alkyl, NH₂, COOR* (R* is hydrogen or C₁₋₆ alkyl), dimethylamino, acetanilide, amide, or C₆₋₁₀ aryl, provided that X cannot be CF₃SO₃, or iodo and bromo when it is halo;

comprising reacting a compound of formula 2:

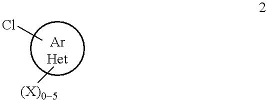

with a compound of formula 3:

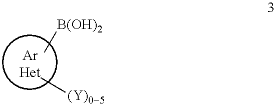

in the presence of a heterogeneous palladium catalyst and a base, a polar aprotic solvent to produce a compound of formula I, wherein wherein X and Y are as previously defined.

This and other aspects of the invention will be realized upon complete review of the specification.

### DETAILED DESCRIPTION OF THE INVENTION

The invention is described herein in detail using the terms defined below unless otherwise specified.

The term “alkyl” refers to a monovalent alkane (hydrocarbon) derived radical containing from 1 to 10 carbon atoms unless otherwise defined. It may be straight, branched or cyclic. Preferred alkyl groups include methyl, ethyl, propyl, isopropyl, butyl, t-butyl, cyclopentyl and cyclohexyl. When substituted, alkyl groups may be substituted with up to four substituent groups, selected from CF₃, C₁₋₆ alkoxy, NO₂, CN, halo, C₁₋₆ alkyl, NH₂, and COOR*, at any available point of attachment. When the alkyl group is said to be substituted with an alkyl group, this is used interchangeably with “branched alkyl group”.

Cycloalkyl is a specie of alkyl containing from 3 to 15 carbon atoms, without alternating or resonating double bonds between carbon atoms. It may contain from 1 to 4 rings which are fused.

Aryl refers to aromatic rings e.g., phenyl, substituted phenyl and the like, as well as rings which are fused, e.g., naphthyl, phenanthrenyl and the like. An aryl group thus contains at least one ring having at least 6 atoms, with up to five such rings being present, containing up to 22 atoms therein, with alternating (resonating) double bonds between adjacent carbon atoms or suitable heteroatoms. The preferred aryl groups are phenyl, naphthyl and phenanthrenyl. Aryl groups may likewise be substituted as defined. Preferred substituted aryls include phenyl and naphthyl.

The term “heteroaryl” refers to a monocyclic aromatic hydrocarbon group having 5 or 6 ring atoms, or a bicyclic aromatic group having 8 to 10 atoms, containing at least one heteroatom, O, S or N, in which a carbon or nitrogen atom is the point of attachment, and in which one or two additional carbon atoms is optionally replaced by a heteroatom selected from O or S, and in which from 1 to 3 additional carbon atoms are optionally replaced by nitrogen heteroatoms, said heteroaryl group being optionally substituted as described herein. Examples of this type are pyrrole, pyridine, oxazole, thiazole and oxazine. Additional nitrogen atoms may be present together with the first nitrogen and oxygen or sulfur, giving, e.g., thiadiazole.

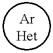

represent a molecule which is a C₆₋₁₀ aryl or C₅₋₁₀ heteroaryl, said aryl or heteroaryl optionally substituted as indicated herein.

Suitable polar aprotic solvents are N,N-dimethylacetamide (DMA), N,N-dimethylformamide (DMF), 1-methyl-2-pyrrolidinone (NMP), Dioxane, ethylene glycol dimethyl ether (DME), diethoxymethane (DEM), tetrahydrofuran (THF) and the like.

X and Y are suitable electron-withdrawing or electron-donating substituents such as hydrogen, CF₃, C₁₋₆ alkoxy, NO₂, CN, halo, C₁₋₆ alkyl, NH₂, COOR*, dimethylamino, acidanilide, amide, C₆₋₁₀ aryl and the like, provided that X cannot be CF₃SO₃, or iodo and bromo when it is halo.

Suitable aryl and heteroaryl chlorides are those in which the aryl or heteroaryl is pyridine, quinolines, thiophene, furan, acetanilide, benzaldehyde, said pyridine, thiophene, quinoline, and furan optionally substituted with 1-3 groups of halo or C₁₋₆ alkyl. Preferred aryl and heteroaryl chlorides are Chlrobenzene, 2-chloropyridine, chlorotoluene, 4-chloropyridine, 4-chlorobenzotrifluoride, 4-chlorobenzonitrile, chlorobenzonitrite, 1-chloro-4-nitrobenzene, and 4′-chloroacetophenone.

Suitable boronic acids are aryl or heteroaryl boronic acids which are substituted with C₆₋₁₀ aryl, methoxy, NO₂, CF3, CHO, amino, CN, COMe, COOH, or C₁₋₆ alkyl. Preferred boronic acids are phenylboronic acid, 4-methoxyphenylboronic acid, 4-methylphenylboronic acid, 4-chlorophenylboronic acid and 4-nitrophenylboronic acid.

The term “heteroatom” means O, S or N, selected on an independent basis.

Halogen and “halo” refer to bromine, chlorine, fluorine and iodine.

Suitable bases include trialkylamines such as triethylamine, trimethylamine, ethyldimethylamine, tri-n-propylamine and the like, 1,8-diazabicyclo[5.4.0.]undec-7-ene (DBU), pyridine, lutidine, collidine, 4-dimethylaminomethyl-pyridine, inorganic carbonates and bicarbonates such as sodium carbonate, sodium bicarbonate, potassium bicarbonate, potassium carbonate, and the like and tartrates such as potassium sodium tartrate, potassium tartrate, potassium bitartrate, sodium tartrate, sodium bitartrate and the like. Preferable bases are pyridine, potassium sodium tartrate, sodium carbonate, sodium bicarbonate, potassium bicarbonate, or potassium carbonate; most preferably is potassium carbonate.

Suitable heterogeneous catalyst are those which contain a palladium (Pd) source including those that are finely dispersed palladium with or without a solid support; or finely dispersed palladium stabilized by organic polymers such as poly(vinylpyrrolidinones), poly(vinyl alcohol) and poly (methyl vinyl ether).

In the case of finely dispersed palladium on a solid support, this includes palladium supported on carbon (Pd/C), silica, alumina, titania, and mesopourous or zeolitic materials. The state of the palladium can be in a reduced or non-reduced form in which case it can be reduced in situ with any suitable reducing agents including aryl boronic acids, potassium or sodium formate, hydrogen, borohydride reagents, silanes, aluminum hydride reagents, hydrazine and the like. In the case of finely dispersed palladium without a solid support this includes finely dispersed palladium metal (Pd Black) and finely dispersed palladium generated from homogeneous palladium sources (such as palladium acetate) by action of a suitable reducing agent including potassium or sodium formate, hydrogen, borohydride reagents, silanes, aluminum hydride reagents, hydrazine and the like. In the case of finely dispersed palladium stabilized by organic polymers, this includes colloidal palladium stabilized by organic polymers such as poly(vinylpyrrolidinones), poly(vinyl alcohol) and poly (methyl vinyl ether).

The reaction is generally carried out using a polar aprotic solvent such as N,N-dimethylacetamide (DMA), N,N-dimethylformamide (DMF), N-methylpyrrolidinone (NMP), dioxane, ethylene glycol dimethyl ether (DME), diethoxymethane (DEM), tetrahydrofuran (THF) and the like. Alternatively, the reaction is carried out in an appropriate combination of one or more of the above solvents with water. Preferable solvents are NMP or DMA in combination with water. When solvent is combined with water the ratio of solvent to water is in the range of about 30:0.5 to about 5:0.5, preferably from about 25:1 to about 5:1 and most preferably from about 20:1 to about 10:1.

In particular, processes of interest are those described above wherein the electron-withdrawing substituents are selected from the group consisting of CF₃, COR″, NO₂, and CN, wherein R″ is C₁₋₆ alkyl.

Other processes of interest are those described above wherein the electron-donating substituents are selected from the group consisting of OR* and C₁₋₆ alkyl, wherein R* is hydrogen or C-₁₋₆ alkyl.

The process of the present invention is illustrated by the following generic scheme:

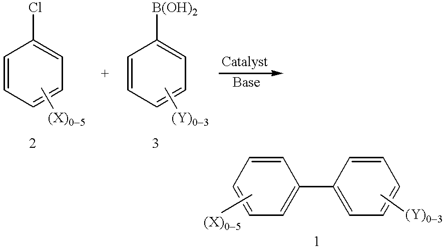

With reference to Flow Sheet A, the compounds used in the synthesis of the compounds of the present invention have, in some cases, been described in the chemical literature. One skilled in the art can adapt a previously published synthesis of an analogous compound to prepare the requisite compound in a straightforward manner without undue experimentation.

In general, the cross coupling reaction can be accomplished by reacting the compound of formula 2 and the compound of formula 3 in a solvent such as N,N-dimethylacetamide (DMA), N,N-dimethylformamide (DMF), N-methylpyrrolidinone (NMP), dioxane and the like or a combination of the above with water, preferably NMP or DMA in combination with water, in the presence of a base such as inorganic carbonates and bicarbonates such as sodium carbonate, sodium bicarbonate, potassium bicarbonate, potassium carbonate, and the like and tartrates such as potassium sodium tartrate, potassium tartrate, potassium bitartrate, sodium tartrate, sodium bitartrate and the like, preferably potassium carbonate and a heterogeneous palladium catalyst such as Pd/C at a temperature between about 35° C. and about 120° C., preferably about 50° C. to about 100° C., and most preferably about 75° C. to about 85° C., followed by an appropriate workup and isolation procedure familiar to those skilled in the art to yield the compounds of I. When solvent is combined with water the ratio of solvent to water is in the range of about 30:0.5 to about 5:0.5, preferably from about 25:1 to about 5:1, more preferably from about 20:1 to about 10:1 and most preferably about 20:1.

The preferable palladium catalyst system is one with an optimal palladium level of about 0.1 mol % to about 15 mol %, preferably about 3 mol % to about 10 mol % with respect to the aryl chloride.

The final product may be characterized structurally by standard techniques such as NMR, IR, MS, and UV. For ease of handling, the final product, if not crystalline, may be lyophilized from water to afford an amorphous, easily handled solid.

The compounds of the present invention are valuable tools for the synthesis of drug intermediates, for example antibacterial agents.

The invention is further described in connection with the following non-limiting examples.

### EXAMPLE 1

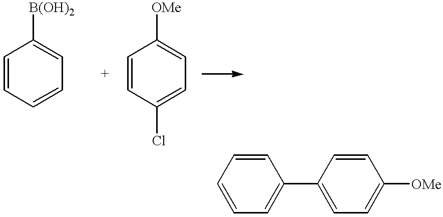

Cross-coupling of 4-chloroanisole with Phenylboronic Acid:

To a clean 40 ml Schlenk tube is added 85 mg (5 mol % Pd based on 4-chloroanisole) of 5 wt % Pd on carbon (PMC type 1610C, 1.72% water), 119 mg phenylboronic acid (0.96 mmol), 221 mg K₂CO₃ (1.6 mmol), 5 ml of N-methylpyrrolidinone and 0.25 ml of distilled water. A magnetic stir bar is added, the schlenk tube sealed with a rubber septum and inerted with four vacuum/argon purge cycles. The 4-chloroanisole, 0.8 mmol (c.a. 100 ul), is then added by syringe, and the Schlenk tube is inerted with four vacuum/argon cycles, placed in an 80° C. oil bath, and stirred for 24 h. The reaction mixture is then filtered and the catalyst cake was with acetonitrile. The filtrate is diluted to 1 liter of acetonitrile, and the yield determined by HPLC comparision with authetic product. In this case the yield was determined to be 33% by HPLC assay. All substrates and solvents are used as purchased from Aldrich without further purification.

### EXAMPLE 2

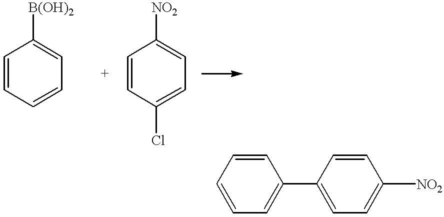

Cross-coupling of 4-chloro-1-nitrobenzene with Phenylboronic Acid:

The same procedure was used in this case except 126 mg (0.8 mmol) of 4-chloro-1-nitrobenzene was used in place of 4-chloroanisole. In this case the yield was determined to be 93% by HPLC assay. All substrates and solvents are used as purchased from Aldrich without further purification.

### EXAMPLE 3

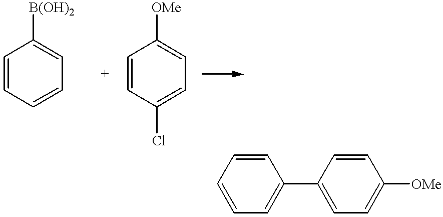

Cross-coupling of 4-chloroanisole with Phenylboronic Acid:

To a clean 40 ml Schlenk tube is added 85.6 mg (5 mol % Pd based on 4-chloroanisole) of 5 wt % Pd on carbon (PMC type 1610C, 1.72% water), 120.3 mg phenylboronic acid (0.987 mmol), 223 mg K₂CO₃ (1.6 mmol), 5 ml of N,N-dimethylacetamide and 0.25 ml of distilled water. A magnetic stir bar is added, the Schlenk tube sealed with a rubber septum and inerted with four vacuum/argon purge cycles. The 4-chloroanisole, 0.8 mmol (c.a. 100 ul), is then added by syringe, and the Schlenk tube is inerted with four vacuum/argon cycles, placed in an 80° C. oil bath, and stirred for 24 h. The reaction mixture is then filtered and the catalyst cake washed with acetonitrile. The filtrate is diluted to 1 liter of acetonitrile, and the yield determined by HPLC comparision with authetic product. In this case the yield was determined to be 29% by HPLC assay. All substrates and solvents are used as purchased from Aldrich without further purification.

### EXAMPLE 4

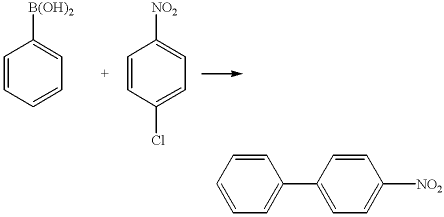

Cross-coupling of 1-chloro-4-nitrobenzene with Phenylboronic Acid:

The same procedure was used in this case except 121.3 mg (0.77 mmol) of 1-chloro-4-nitrobenzene was used in place of 4-chloroanisole. In this case the yield was determined to be 93% by HPLC assay. All substrates and solvents are used as purchased from Aldrich without further purification.

### EXAMPLE 5

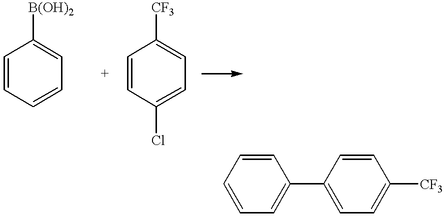

Cross-coupling of 4-chlorotrifluoromethylbenzene with Phenylboronic Acid:

The same procedure was used in this case except 105 μL (0.77 mmol) of 4-chlorotrifluoromethylbenzene was used in place of 4-chloroanisole. In this case the yield was determined to be 95% by HPLC assay. All substrates and solvents are used as purchased from Aldrich without further purification.

### EXAMPLE 6

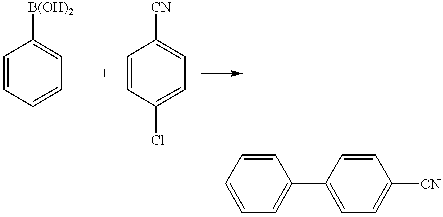

Cross-coupling of 1-chloro-4-cyanobenzene with Phenylboronic Acid:

The same procedure was used in this case except 111 mg (0.79 mmol) of 1-chloro-4-cyanobenzene was used in place of 4-chloroanisole. In this case the yield was determined to be 83% by HPLC assay. All substrates and solvents are used as purchased from Aldrich without further purification.

### EXAMPLE 7

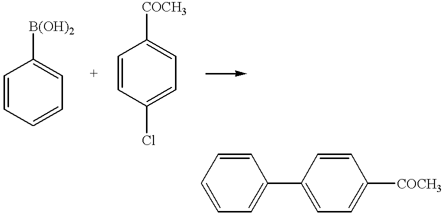

Cross-coupling of 4-chloroacetophenone with Phenylboronic Acid:

The same procedure was used in this case except 125 mg (0.81 mmol) of 4-chloroacetophenone was used in place of 4-chloroanisole. In this case the yield was determined to be 79% by HPLC assay. All substrates and solvents are used as purchased from Aldrich without further purification.

### EXAMPLE 8

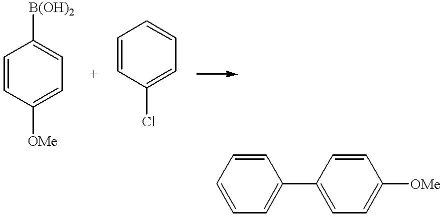

Cross-coupling of chlorobenzene with 4-Methoxyphenylboronic Acid:

The same procedure was used in this case except 89 mg (0.79 mmol) of chlorobenzene and 149.3 mg (0.98 mmol) of 4-methoxyphenylboronic acid. In this case the yield was determined to be 45% by HPLC assay. All substrates and solvents are used as purchased from Aldrich without further purification.

### EXAMPLE 9

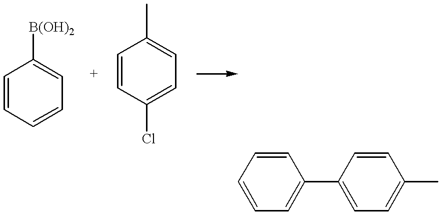

Cross-coupling of 4-chlorotoluene with Phenylboronic Acid:

The same procedure was used in this case except 102 mg (0.80 mmol) of 4-chlorotoluene was used in place of 4-chloroanisole. In this case the yield was determined to be 36% by HPLC assay. All substrates and solvents are used as purchased from Aldrich without further purification.

What is claimed is:

1. A ligandless process for cross-coupling aryl or heteroaryl chlorides with aryl or heteroaryl boronic acids, comprising combining an aryl or heteroaryl chloride and an aryl or heteroaryl boronic acid with a polar aprotic solvent in the presence of a base and heterogeneous palladium catalyst to produce the resulting coupled compound.

2. A process according to claim 1 wherein the aryl or heteroaryl chlorides comprises chlorobenzene, 2-chloropyridine, 4-chloropyridine, 4-chlorobenzotrifluoride, 4-chlorobenzonitrile, 1-chloro-4-nitrobenzene, or 4′-chloroacetophenone and the aryl or heteroaryl boronic acids comprises phenylboronic acid, 4-methoxyphenylboronic acid, 4-methylphenylboronic acid, 4-chlorophenylboronic acid or 4-nitrophenylboronic acid.

3. A process according to claim 1 wherein the heterogeneous palladium catalyst is finely dispersed palladium on a solid support.

4. A process according to claim 3 wherein the solid support comprises carbon (Pd/C), silica, alumina, titania, or mesopourous or zeolitic materials.

5. A process according to claim 1 wherein the heterogeneous palladium catalyst is finely dispersed palladium without a solid support.

6. A process according to claim 5 wherein the finely dispersed palladium is finely dispersed palladium metal (Pd Black) or finely dispersed palladium generated from homogeneous palladium sources such as palladium acetate.

7. A process according to claim 1 wherein the heterogeneous palladium catalyst is finely dispersed palladium (colloidal) stabilized by organic polymers.

8. A process according to claim 1 wherein the polar aprotic solvent comprises N,N-dimethylacetamide (DMA), N,N-dimethylformamide (DMF), N-methylpyrrolidinone (NMP), dioxane, ethylene glycol dimethyl ether (DME), diethoxymethane (DEM), tetrahydrofuran (THF) or a combination of one or more of the above with water is added.

9. A process according to claim 8 wherein the solvent is NMP or DMA in combination with water.

10. A process according to claim 1 wherein the solvent is combined with water and the ratio of solvent to water is about 30:0.5 to about 5:0.5.

11. A process according to claim 10 wherein the ratio of solvent to water is from about 25:1 to about 5:1.

12. A process according to claim 11 wherein the ratio of solvent to water is from about 20:1 to about 10:1.

13. A process according to claim 1 wherein the base comprises triethylamine, trimethylamine, ethyldimethylamine, tri-n-propylamine, 1,8-diazabicyclo[5.4.0.]undec-7-ene (DBU), pyridine, lutidine, collidine, 4-dimethylaminomethyl-pyridine, sodium carbonate, sodium bicarbonate, potassium bicarbonate, potassium carbonate, potassium sodium tartrate, potassium tartrate, potassium bitartrate, sodium tartrate, or sodium bitartrate.

14. A process according to claim 13 wherein the base is sodium or potassium phosphate, cesium fluride, sodium t-butoxide, pyridine, potassium sodium tartrate, sodium carbonate, sodium bicarbonate, potassium bicarbonate, or potassium carbonate.

15. A ligandless process for making a compound of formula I: 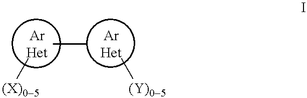
is disclosed wherein:
Ar represents C₆₋₁₀ o aryl;
Het represents C₅₋₁₀ heteroaryl;
X and Y independently represent hydrogen, CF₃, C₁₋₆ alkoxy, NO₂, CN, halo, C₁₋₆ alkyl, NH₂, COOR* (R* is hydrogen or C₁₋₆ alkyl), dimethylamino, acetanilide, amide, or C₆₋₁₀ aryl, provided that X cannot be CF₃SO₃, or iodo and bromo when it is halo;
comprising reacting a compound of formula 2: 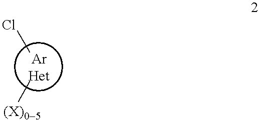
with a compound of formula 3: 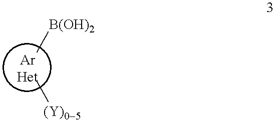
boronates, —B(OR)₂, are fine too. in the presence of a heterogeneous palladium catalyst and a base, a polar aprotic solvent to produce a compound of formula I, wherein wherein X and Y are as previously defined.

16. A process according to claim 15 wherein the aryl or heteroaryl chlorides are selected from the group consisting of chlorobenzene, 2-chloropyridine, 4-chloropyridine, 4-chlorobenzotrifluoride, 4-chlorobenzonitrile, 1-chloro-4-nitrobenzene, and 4′-chloroacetophenone and the aryl or heteroaryl boronic acids are selected from the group consisting of boronic acids are phenylboronic acid, 4-methoxyphenylboronic acid, 4-methylphenylboronic acid, 4-chlorophenylboronic acid and 4-nitrophenylboronic acid.

17. A process according to claim 15 wherein the heterogeneous palladium catalyst is finely dispersed palladium on a solid support.

18. A process according to claim 17 wherein the solid support is selected from the group consisting of carbon (Pd/C), silica, alumina, titania, and mesopourous or zeolitic materials.

19. A process according to claim 15 wherein the heterogeneous palladium catalyst is finely dispersed palladium without a solid support.

20. A process according to claim 19 wherein the finely dispersed palladium is finely dispersed palladium metal (Pd Black) or finely dispersed palladium generated from homogeneous palladium sources such as palladium acetate.

21. A process according to claim 15 wherein the heterogeneous palladium catalyst is finely dispersed palladium (colloidal) stabilized by organic polymers.

22. A process according to claim 15 wherein the polar aprotic solvent is selected from the group consisting of N,N-dimethylacetamide (DMA), N,N-dimethylformamide (DMF), N-methylpyrrolidinone (NMP), dioxane ethylene glycol dimethyl ether (DME), diethoxymethane (DEM), tetrahydrofuran (THF) or a combination of one or more of the above with water is added.

23. A process according to claim 22 wherein the solvent is NMP or DMA in combination with water.

24. A process according to claim 15 wherein the solvent is combined with water and the ratio of solvent to water is about 30:0.5 to about 5:0.5.

25. A process according to claim 24 wherein the ratio of solvent to water is from about 25:1 to about 5:1.

26. A process according to claim 25 wherein the ratio of solvent to water is from about 20:1 to about 10:1.

27. A process according to claim 15 wherein the base is selected from the group consisting of triethylamine, trimethylamine, ethyldimethylamine, tri-n-propylamine, 1,8-diazabicyclo[5.4.0.]undec-7-ene (DBU), pyridine, lutidine, collidine, 4-dimethylaminomethyl-pyridine, sodium carbonate, sodium bicarbonate, potassium bicarbonate, potassium carbonate, potassium sodium tartrate, potassium tartrate, potassium bitartrate, sodium tartrate, and sodium bitartrate.

28. A process according to claim 27 wherein the base is pyridine, potassium sodium tartrate, sodium carbonate, sodium bicarbonate, potassium bicarbonate, or potassium carbonate.
